(19) United States
(12) Patent Application Publication
(10) Pub. No.: US 2024/0307475 A1
(43) Pub. Date: Sep. 19, 2024

(54) Gummy Composition for Relief from Hangover

(71) Applicant: Ricardo Cheaz, Doral, FL (US)

(72) Inventor: Ricardo Cheaz, Doral, FL (US)

(21) Appl. No.: 18/184,470

(22) Filed: Mar. 15, 2023

(51) Int. Cl. A61K 36/73; A61K 31/198; A61K 31/352; A61K 31/51; A61K 36/28; A61K 36/36; A61K 47/12; A61K 47/36

(52) U.S. Cl. CPC A61K 36/73; A61K 31/198; A61K 31/352; A61K 31/51; A61K 36/28; A61K 36/36; A61K 47/12; A61K 47/36

(57) ABSTRACT

A gummy composition for relief from hangover is disclosed. The composition comprising Granulated Sugar 25%, Glucose syrup 27.55%, Purified Water 10.10%, High Methoxyl Pectin 1.80%, Citric acid 1.33%, Food grade Potassium citrate 7.75%, Dihydromyricitin 2.17%, N-Acetyl-Cysteine 15%, Thiamin (B1) 1.25%, Milk thistle extract 2.50%, Green tea extract 2.50%, Prickly pear cactus extract 2.50%, Asian pear juice concentrate 70 Brix 0.50%, Powder Potassium sorbate 0.05%. The the composition may be formed of a candy coated with Carnauba wax 95% and 5% oil.

### BACKGROUND

### Field of Invention

The present invention generally relates to a gummy composition and more particularly, to a gummy composition for relief from hangover.

### Description of Related Art

People drink alcoholic beverages to socialize, celebrate, and relax. An alcoholic beverage contains ethanol, a type of alcohol that acts as a drug and is produced by fermentation of grains, fruits, or other sources of sugar. The consumption of alcoholic beverages in higher doses causes drunkenness, stupor, unconsciousness, or death. Long-term use can lead to an alcohol use disorder, an increased risk of developing several types of cancer, cardiovascular disease, and physical dependence.

For many people, a night of drinking can lead to a painful morning after and the dreaded effects of a hangover. A hangover includes a set of symptoms that occur as a consequence of drinking too much. Typical symptoms include fatigue, weakness, thirst, headache, muscle aches, nausea, stomach pain, vertigo, sensitivity to light and sound, anxiety, irritability, sweating, and increased blood pressure. A hangover can vary from person to person.

Alcohol is the main culprit in a hangover, but other components of alcoholic beverages might contribute to hangover symptoms or make a hangover worse. Congeners are compounds, other than ethyl alcohol, that are produced during fermentation. These substances contribute to the taste and smell of alcoholic beverages. Darker spirits, such as bourbon, which tend to have higher levels of congeners than clear spirits, could worsen hangover symptoms for some people. Sulfites are compounds that are added to wine as preservatives. People who have a sensitivity to sulfites may experience a headache after drinking wine.

Alcohol suppresses the release of vasopressin, a hormone produced by the brain that sends signals to the kidneys causing them to retain fluid. As a result, alcohol increases urination and excess loss of fluids. The dehydration contributes to hangover symptoms such as thirst, fatigue, and a headache. People may fall asleep faster after drinking alcohol, but their sleep is fragmented, and they tend to wake up earlier. This contributes to fatigue, as well as lost productivity. Alcohol directly irritates the lining of the stomach and increases acid release. This can lead to nausea and stomach discomfort. Alcohol increases inflammation in the body. Alcohol metabolism, primarily by the liver, creates the compound acetaldehyde, a toxic, short-lived byproduct, which contributes to inflammation in the liver, pancreas, brain, gastrointestinal tract, and other organs. Because individuals are so different, it is difficult to predict how many drinks will cause a hangover. Any time people drink to intoxication, there is a chance they could have a hangover the next day. Hangovers can be both painful and dangerous. During a hangover, a person's attention, decision-making, and muscle coordination can all be impaired. Also, the ability to perform important tasks, such as driving, operating machinery, or caring for others can be negatively affected. Although many remedies for alleviating hangovers are mentioned on the web and in social media, none have been scientifically proven to be effective. There is no magic potion for beating hangovers and only time can help. A person must wait for the body to finish clearing the toxic byproducts of alcohol metabolism, to rehydrate, to heal irritated tissue, and to restore immune and brain activity to normal. There is no way to speed up the brain's recovery from alcohol use by drinking coffee, taking a shower, or having an alcoholic beverage the next morning will not cure a hangover.

Some people take over-the-counter pain relievers (often acetaminophen) before going to bed to minimize hangovers. It is important to recognize that the combination of alcohol and acetaminophen can be toxic to the liver. Like alcohol, certain over-the-counter pain relievers, including aspirin and ibuprofen, can increase acid release and irritate the lining of the stomach.

Ultimately, the only surefire remedy for a hangover is to avoid drinking or drinking in moderation.

Therefore, there is a need to provide a composition for relief from hangover due to consumption of alcohol or alcoholic beverages.

### SUMMARY

The object of the present invention is to provide a composition for relief from hangovers.

Another object of the invention is to provide a gummy composition to facilitate an alcohol hangover remedy.

The other object of the invention is to provide a composition of matter that ameliorates certain negative effects of excess alcohol consumption.

Present invention provides a gummy composition for relief from hangover, the composition comprising Granulated Sugar 25%, Glucose syrup 27.55%, Purified Water 10.10%, High Methoxyl Pectin 1.80%, Citric acid 1.33%, Food grade Potassium citrate 7.75%, Dihydromyricitin 2.17%, N-Acetyl-Cysteine 15%, Thiamin (B1) 1.25%, Milk thistle extract 2.50%, Green tea extract 2.50%, Prickly pear cactus extract 2.50%, Asian pear juice concentrate 70 Brix 0.50%, Powder Potassium sorbate 0.05%.

In the embodiment, the glucose syrup has dextrose equivalent of 42. Further, the Dihydromyricitin is extracted from leaf of Ampelopsis grossendentata.

In the embodiment, the Citric acid used in the composition is 50% weight by volume solution.

In the embodiment, the Milk thistle extract is obtained from seeds of Silybum maranium.

In the embodiment, the green tea extract is obtained from leaf of Camellia sinensis.

In the embodiment, the prickly pear cactus extract from leaf of Opuntia ficas-indica.

In the embodiment, the composition further comprises alcohol dehydrogenase (ADH) and aldehyde dehydrogenase (ALDH).

In an embodiment, the composition is in the form of candy coated with Carnauba wax 95% and 5% oil.

The preceding is a simplified summary to provide an understanding of some embodiments of the present invention. This summary is neither an extensive nor exhaustive overview of the present invention and its various embodiments. The summary presents selected concepts of the embodiments of the present invention in a simplified form as an introduction to the more detailed description presented below. As will be appreciated, other embodiments of the present invention are possible utilizing, alone or in combination, one or more of the features set forth above or described in detail below.

### DETAILED DESCRIPTION

The following description includes the preferred best mode of one embodiment of the present invention. It will be clear from this description of the invention that the invention is not limited to these illustrated embodiments, but the invention also includes a variety of modifications and embodiments thereto. Therefore, the present description should be seen as illustrative and not limiting. While the invention is susceptible to various modifications and alternative constructions, it should be understood, that there is no intention to limit the invention to the specific form disclosed, but, on the contrary, the invention is to cover all modifications, alternative constructions, and equivalents falling within the spirit and scope of the invention as defined in the claims.

In any embodiment described herein, the open-ended terms “comprising,” “comprises,” and the like (which are synonymous with “including,” “having” and “characterized by”) may be replaced by the respective partially closed phrases “consisting essentially of,” consists essentially of,” and the like or the respective closed phrases “consisting of,” “consists of, the like.

As used herein, the singular forms “a”, “an”, and “the” designate both the singular and the plural, unless expressly stated to designate the singular only.

Present invention provides a gummy composition for relief from hangover, the composition comprising Granulated Sugar 25%, Glucose syrup 27.55%, Purified Water 10.10%, High Methoxyl Pectin 1.80%, Citric acid 1.33%, Food grade Potassium citrate 7.75%, Dihydromyricitin 2.17%, N-Acetyl-Cysteine 15%, Thiamin (B1) 1.25%, Milk thistle extract 2.50%, Green tea extract 2.50%, Prickly pear cactus extract 2.50%, Asian pear juice concentrate 70 Brix 0.50%, Powder Potassium sorbate 0.05%.

In the embodiment, the glucose syrup has dextrose equivalent of 42. Further, the Dihydromyricitin is extracted from leaf of Ampelopsis grossendentata. Dihydromyricetin is also known as Ampelopsin, a flavonoid component counteracting acute alcohol intoxication. In the embodiment, the Citric acid used in the composition is 50% weight by volume solution.

The High Methoxyl Pectin has setting properties to the composition.

N-Acetyl-Cysteine enzymatically prevents the hepatocellular toxic effects associated with hangover symptoms. Vitamin B1 (Thiamine) helps in the functioning of brain and nervous system and aid body in getting over the hangover easily.

The asian pear juice concentrate lowers level of blood alcohol. Powder Potassium sorbate used as preservative in the embodiment.

In the embodiment, the Milk thistle extract is obtained from seeds of Silybum maranium has protective effect on liver preventing damage.

In the embodiment, the green tea extract is obtained from leaf of Camellia sinensis. It helps to reduce common hangover symptoms, including nausea, hangover headaches, and lessened ability to focus and concentrate.

In the embodiment, the prickly pear cactus extract from leaf of Opuntia ficas-indica. It reduces the severity of hangovers.

In an embodiment, the composition is in the form of candy coated with Carnauba wax 95% and 5% oil.

While the invention has been described in connection with what is presently considered to be the most practical and various embodiments, it is to be understood that the invention is not to be limited to the disclosed embodiments, but on the contrary, is intended to cover various modifications and equivalent arrangements included within the spirit and scope of the appended claims.

This written description uses examples to disclose the invention, including the best mode, and also to enable any person skilled in the art to practice the invention, including making and using any devices or systems and performing any incorporated methods. The patentable scope the invention is defined in the claims, and may include other examples that occur to those skilled in the art. Such other examples are intended to be within the scope of the claims if they have structural elements that do not differ from the literal language of the claims, or if they include equivalent structural elements within substantial differences from the literal languages of the claims.

What is claimed is:

1. A gummy composition for relief from hangover, the composition comprising
Asian pear juice concentrate 70 Brix 0.50%;
Purified Water 10.10%;
High Methoxyl Pectin 1.80%;
Citric acid 1.33%;
Food grade Potassium citrate 7.75%;
Dihydromyricitin 2.17%;
N-Acetyl-Cysteine 15%;
Thiamin (B1) 1.25%;
Milk thistle extract 2.50%;
Green tea extract 2.50%; and
Prickly pear cactus extract 2.50%.

2. The composition as claimed in claim 1, wherein the composition has granulated sugar 25% and glucose syrup 27.55%.

3. The composition as claimed in claim 1, wherein the composition further comprises alcohol dehydrogenase (ADH) and aldehyde dehydrogenase (ALDH).

4. The composition as claimed in claim 1, wherein the glucose syrup has dextrose equivalent of 42.

5. The composition as claimed in claim 1, wherein Dihydromyricitin is extracted from leaf of Ampelopsis grossendentata.

6. The composition as claimed in claim 1, wherein the Citric acid is 50% w/v solution.

7. The composition as claimed in claim 1, wherein the composition is in the form of candy coated with Carnauba wax 95% and 5% oil.

8. The composition as claimed in claim 1, wherein Milk thistle extract is obtained from seed of Silybum maranium.

9. The composition as claimed in claim 1, wherein green tea extract is obtained from leaf of Camellia sinensis.

10. The composition as claimed in claim 1, wherein prickly pear cactus extract from leaf of Opuntia ficas-indica.

11. The composition as claimed in claim 1, wherein 0.05% Potassium sorbate is added as preservative.
